(19) United States
(12) Patent Application Publication
(10) Pub. No.: US 2017/0124747 A1
(43) Pub. Date: May 4, 2017

(54) SYNTHETICALLY FABRICATED AND CUSTOM FITTED DRESSWARE

(71) Applicant: Edward KNOWLTON, Indian Wells, CA (US)

(72) Inventor: Edward KNOWLTON, Indian Wells, CA (US)

(21) Appl. No.: 15/284,082

(22) Filed: Oct. 3, 2016

Related U.S. Application Data
(60) Provisional application No. 62/236,546, filed on Oct. 2, 2015.

(51) Int. Cl. G06T 11/60; G06T 13/40; A41H 3/00; A61F 2/50; A41D 31/00; G06K 9/00; G06T 17/20

(52) U.S. Cl. CPC G06T 11/60; G06K 9/00362; G06T 13/40; G06T 17/20; A61F 2/5046; A41D 31/00; A41H 3/007; A61F 2002/505; A61F 2/52

(57) ABSTRACT

Embodiments are configured for capturing digital data representing skin of a subject. An avatar representing the skin is generated from the digital data. An enhanced avatar is generated by digitally revising a contour of the skin of the body. A digital garment configured to enhance contours of the skin of the body is rendered from the enhanced avatar. The digital garment comprises material having numerous zones, and each zone is configured with material comprising a contouring force and/or a pigment pattern corresponding to a region of the skin corresponding to the zone. A corrective garment is generated from the digital garment, and the corrective garment is configured for wear to cover one or more regions of the skin of the body. The corrective garment is configured to aesthetically enhance the body.

### RELATED APPLICATION

This application claims the benefit of U.S. patent application Ser. No. 62/236,546, filed Oct. 2, 2015.

### TECHNICAL FIELD

The embodiments herein relate to medical systems, instruments or devices, and methods and, more particularly, to digital capture and rendering of the human body for use in generating custom fitted, synthetically fabricated dressware for body contouring, and data and processing relating to the synthetic garments.

### BACKGROUND

Conventional computer-assisted methods and systems for garment design and manufacture, particularly automated using software, automate garment and fashion definition and production. Configurable garments available using these methods and systems include ornamental elements, pattern displays, and personal identifiers and wireless sensor electronics. However, there is a need for systems and methods configured to digitally capture and render data of an individual's body for use in generating custom fitted, synthetically fabricated garments or dressware for body contouring.

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### BRIEF DESCRIPTION OF THE DRAWINGS

FIG. 1 shows a digital capture (3D) of a body, under an embodiment.

FIG. 2A shows a rendered wire mesh on the 3D body capture, under an embodiment.

FIG. 2B shows the wire mesh corresponding to the digital 3D capture, under an embodiment.

FIG. 3 is a system for generating the Dresswear or Skinwear, under an embodiment.

FIG. 4 is a flow diagram for generating the Dresswear or Skinwear, under an embodiment.

FIG. 5 shows the wire mesh corresponding to the digital 3D capture (left), and the wire mesh with selective zoning in the bra zone and girdle zone (right), under an embodiment.

FIG. 6 shows the digital 3D capture of a body without the wire mesh garment (left), and the 3D capture of the body with the wire mesh with selective zoning in the bra zone and girdle zone (right), under an embodiment.

FIG. 7 shows the body with the Skinwear with camouflage (left), and the body with the Skinwear without camouflage (right), under an embodiment.

FIG. 8 shows custom patterned Skinwear, under an embodiment.

FIG. 9 shows a custom patterned Skinwear ensemble, under an embodiment.

FIG. 10 shows custom patterned Skinwear configured for applications involving traumatic scar and kinetic contour deformities, under an embodiment.

FIG. 11 shows custom patterned Skinwear configured for applications involving post mastectomy chest deformities, under an embodiment.

### DETAILED DESCRIPTION

Embodiments include systems and methods configured for capturing digital data representing skin of a body or subject, and generating from the digital data an avatar representing the skin of the body. A digital garment is rendered using data of the avatar. The digital garment is configured to match contours of the skin of the body. A custom-fitted garment configured for wear by the body is generated from the digital garment a custom-fitted garment.

Embodiments include systems and methods configured for capturing digital data representing skin of a subject. An avatar representing the skin of the body is generated from the digital data. An enhanced avatar is generated by digitally revising a contour of the skin of the body. A digital garment configured to enhance contours of the skin of the body is rendered from the enhanced avatar. The digital garment comprises material having numerous zones, and each zone is configured with material comprising a contouring force and/or a pigment pattern corresponding to a region of the skin corresponding to the zone. A corrective garment is generated from the digital garment, and the corrective garment is configured for wear to cover one or more regions of the skin of the body. The corrective garment is configured to aesthetically enhance the body.

Technology is applied to or used in the garment or fashion industry, for example in computer-aided processes for design and modeling of garments or clothing. Examples of such technology are found in the following U.S. Pat. Nos. 8,930,012; 8,185,231; 8,116,895; 8,065,029; 6,882,897; 6,725,124; 6,564,118; 6,473,671; 5,850,222; 3,992,903. Another example of technology applied to the garment or fashion industry is found in “Smart Body—Ergonomic Seamless Sportswear Design and Development,” by O. Troynikov, The Body: Connections with Fashion, Conference Proceedings 2008, International Foundation of Fashion Technology Institutes (IFFTI).

Furthermore, medical simulation systems of embodiments described herein are based on biomechanical information of tissue modeling, because representing physical phenomena and, more specifically, the realistic modeling of tissue systems, improves current medical simulation systems and enlarges the set of applications and the credibility of medical simulation. Realistic tissue simulation is performed using factors of tissue modeling combined with mechanical modeling and computer processing. Examples relating to these types of modeling are found in the following: “Toward Realistic Soft-Tissue Modeling In Medical Simulation,” by H. Delingette, Proceedings of the IEEE, Volume 86, Issue 3, March 1998; “Tensor Analysis and Nonlinear Tensor Functions,” by Yu. I. Dimitrienko, Springer Science+Business Media Dordrecht, 2002. The modeling includes, for example, spring models (used extensively for simulating elasticity of soft tissue), finite element models, models describing mathematical factors of a soft conformable filler with a semi-elastic membrane on a 3D deformable contour, models describing mathematical factors of a semi-elastic layer (for both synthetic fabrics and skin) affecting a 3D deformable contour including the effects of a soft filler of variable deformability and viscosity, models of skin elasticity rebound including properties of skin that reduce rebound from displacement such as aging with thinning of the dermis and loss of elastin fibers, and models of the physical properties concerning turgidity of soft tissue and its relationship to deformation (resistance to conformability), but is not so limited.

Moreover, the advent of synthetic fibers has brought an era of form and fashion that has no historical equivalent. With software control, the potential for true customization exists for a large variety of consumer products. Digital capturing and software rendering enables the detailed analysis of the consumer's three soft tissue embodiment. Based on this data set, the capability exists to manufacture a custom fitted Dresswear that is formatted by that individual data set. Another product is a closely-contouring synthetic fabric that mimics the individual's own three dimensional skin envelope. The Aesthetic and Reconstructive capabilities of this new “Skinwear” promise a novel alternative to skin laxity, lipdystrophy induced contour deformities, skin scar deformities and three dimensional losses of symmetry such as post-mastectomy chest deformities.

Embodiments described herein include the fabrication of a conformable synthetic garment without visually apparent seaming that contours on the basis of a digital capture, software rendering, re-rendering of a person's skin soft tissue embodiment. The re-rendering and subsequent garment fabrication vectors and conforms the person's per-existing skin soft tissue embodiment into a more aesthetically enhanced embodiment. The systems of embodiments include a database comprising a dataset of zoned parameters of a digitally rendered wire mesh with dataset of measured skin embodiment parameters that renders a digital mapping for effecting the custom fabrication of a garment that creates an aesthetically enhanced skin/soft tissue embodiment of the person.

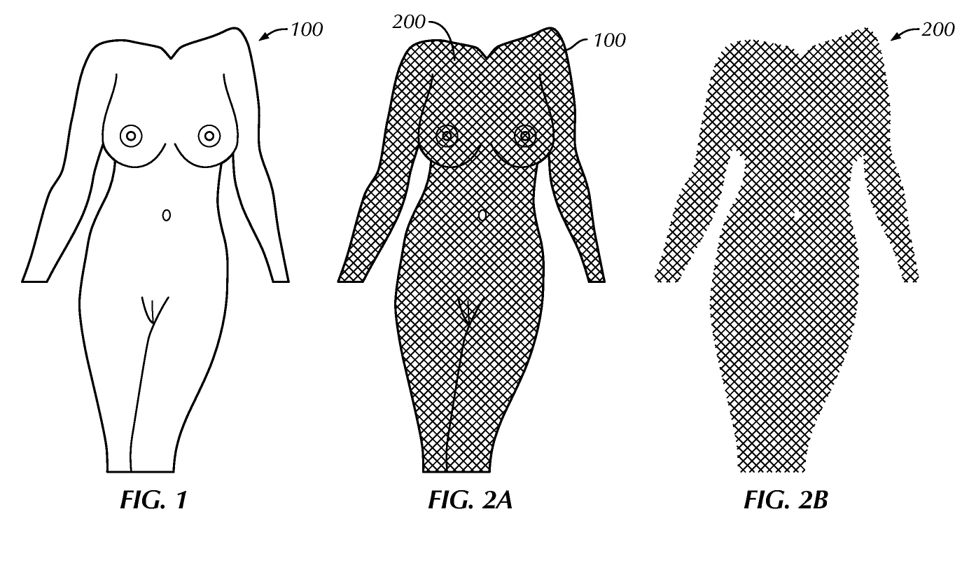

Embodiment herein involve use of three-dimensional (3D) and/or four-dimensional (4D) rendering software to develop and produce new custom garments that more accurately follow the unique contours of the individual's embodiment. The digital capture of the person's 3D embodiment enables rendering of a digital wire mesh embodiment and that digital wire mesh can then be re-rendered per fashion specification by the customer. FIG. 1 shows a digital capture (3D) 100 of a body, under an embodiment. FIG. 2A shows a rendered wire mesh 200 on the 3D body capture 100, under an embodiment. FIG. 2B shows the wire mesh 200 corresponding to the digital 3D capture, under an embodiment. This capability also has significant benefits for the unique needs of a patient with a soft tissue contour deformity as described in detail herein.

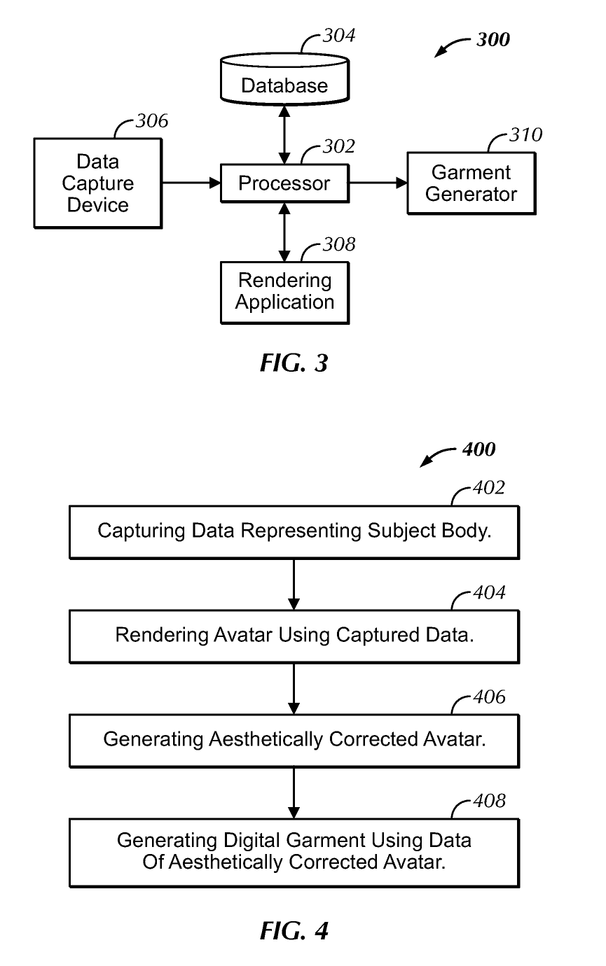

FIG. 3 is a system 300 for generating the Dresswear or Skinwear, under an embodiment. The system includes a processor 302 coupled to a memory comprising a database 304. A data capture device 306 or other detector is coupled to the processor and configured to capture digital data representing skin of a body. A rendering application 308 is configured to execute or run on the processor. The rendering application is configured to generate from the digital data an avatar representing the skin of the body, and generate an enhanced avatar by digitally revising a contour of the skin of the body. The rendering application 308 is configured to render from the enhanced avatar a digital garment configured to enhance contours of the skin of the body. The digital garment comprises material having numerous zones, each configured with material comprising a a contouring force and/or a pigment pattern corresponding to a region of the skin corresponding to the zone. A corrective garment is generated from the digital garment, and in an embodiment the corrective garment is generated by a garment generator 310 that is one or more of a component of the system 300 and remote to the system (e.g., third party manufacturer, etc.). The corrective garment is configured for wear to cover one or more regions of the skin of the body to aesthetically enhance the body, as described in detail herein.

FIG. 4 is a flow diagram 400 for generating the Dresswear or Skinwear, under an embodiment. The method comprises digital capture of data of the subject's skin embodiment 402. The digital capture is rendered into an avatar, which in an embodiment includes a digital wire mesh 404. In an embodiment, the rendering is performed in multiple 3D views including frontal (A-P), Lateral and Oblique Views. Rendering can also be performed in 4D to provide an animated format. Alternatively, 4D can be applied to the effect of time on the human embodiment. The effects of aging and wound healing are depicted over a period of time as a non-animated 4D format.

The method comprises re-rendering the digital avatar (e.g., wire mesh) into an aesthetically corrected 3D digital wire mesh for transfer to a fabricator 406. The final re-rendering of the corrected 3D digital wire mesh into the aesthetically corrected embodiment of the patient/customer can be generated by texture mapping of the person's skin, but is not so limited. A digital garment is subsequently generated using data of the aesthetically corrected avatar 408.

In an embodiment, digital data of the subject's body is captured. The data capture of an embodiment includes capturing a digital photo or other optical data of the individual's body but is not so limited. Digital data capture also includes the capturing of data using medical device(s) known in the art for such capture. The digital data comprises data of physical properties of at least one of the skin and soft tissue underlying the skin. For example, the digital data comprises one or more of pigment data, pigment pattern data, tone data, color data, texture data, elasticity data, laxity data, deformity data, wrinkling data, scar data, and burn data of at least one of the skin and soft tissue underlying the skin.

The digital data collected (e.g., photo, medical device, etc.) is rendered into a digital wire mesh configured as a format for fabricating a custom Dresswear that more accurately follows the individual's skin/soft tissue embodiment. With this rendering process, a custom made garment is precisely fitted for each subject. In another application, the rendered digital wire mesh is subsequently re-rendered into a contour-enhanced version. This re-rendered digital wire mesh can also act as a format for the development of a Skinwear garment that provides enhancement of the subject's three-dimensional embodiment. Texture mapping of the individual's skin onto the re-rendered wire mesh also provides additional visual input for the fabrication of a corrective Skinwear that more closely simulates the native texture and/or pigment or pigmentary pattern of the individual.

A garment generator or fabricator is used in an embodiment to manufacture or produce the custom garment to unique dimensions and tastes of the customer. For contour enhancement, the synthetic materials of the Dresswear are zoned with variable parameters (e.g., elasticity, thickness, material properties, etc.) to provide aesthetic contour correction. Inward contouring is provided by less flexible synthetic materials that are oriented over anatomic clefts such the vertical midline gluteal cleft and the horizontal inframammary folds. Outward contouring is provided by synthetic materials that are more flexible for outward expansion over convex contours. Variable flexibility is provided with zoning of different synthetic materials or by varying the properties of the materials of various zones (e.g., material properties, thickness of synthetic materials in the Z (depth) axis, etc.). Depth deposition and synthetic component zoning provide seamless detailed contouring of anatomical regions.

Artificial fibers are selectively deposited to form the Skinwear by a computerized reprogrammable fabricator but are not so limited. In another embodiment, 3D printing technology is used with (or as a replacement to) a computerized reprogrammable fabricator. Alternatively, the Skinwear is generated using a mold or molding process (e.g., positive mold, negative mold, thermoplastic mold, mold for thermoplastic Skinwear, etc.). The use of medical devices to make objective measurements of the physical properties of the skin and soft tissue provides more resolution to the re-rendering of the Skinwear garment i.e., manometer for measuring the deformability of the skin envelope and the subcutaneous soft tissue. Skin laxity and elasticity are additional parameters to be entered in the person's dataset.

The mathematical modeling of synthetic fabric with semi-elastic physical properties can also be applied to describe the person's own semi-elastic skin envelope. Determining the conformability of each person's skin/soft tissue envelope and then mapping those differences topographically by anatomical location provides greater resolution to the fabric zoning required to produce an aesthetically contoured skin/soft tissue embodiment.

Another embodiment includes the use of layering (instead of mixing) of synthetic fibers to form a layered and zoned composite fabric that possess selective contouring with a seamless appearance. Layering is performed in the Z-axis of an embodiment to preserve the seamless appearance of the Skinwear, but is not so limited.

In an example embodiment, a subject takes his/her own photos at home and sends/uploads them to a secure website where their skin embodiment is converted/rendered into a digital wire mesh that is used to create a digitally rendered Dresswear. Upon approval by the individual, the custom garment is rendered online into a custom fitted Dresswear. The data set of each approved garment is then sent to a programmable synthetic fabricator for production.

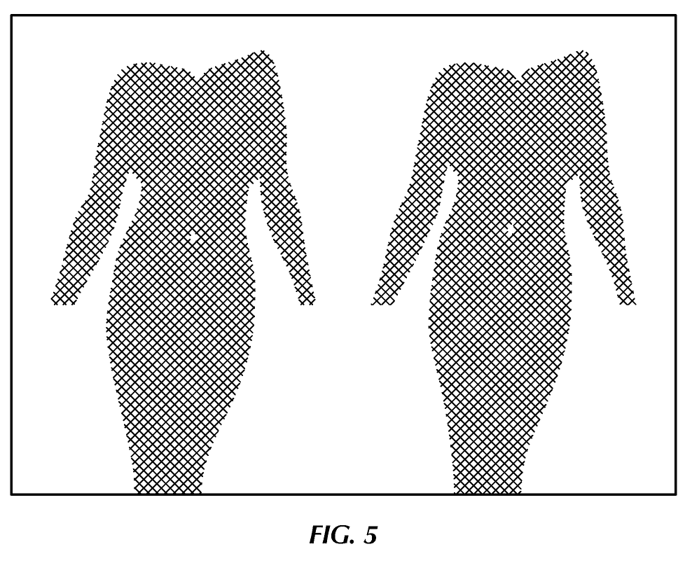

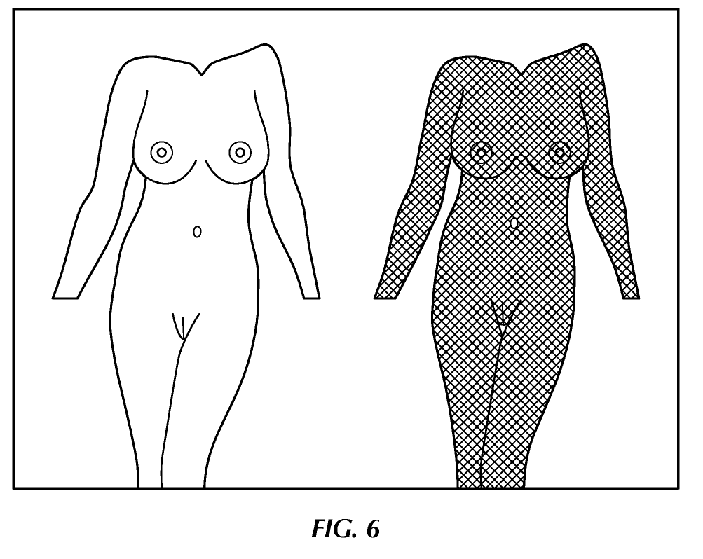

In an alternative example embodiment, the subject's skin embodiment is digitally captured in a “fitting room” at a retail store where a Dresswear consultant (sitting with the customer) renders the customer's skin embodiment into a digital wire mesh that is re-rendered into a corrected 3D digital mesh that is then subsequently formatted onto a digital mosaic zones of synthetic fabrics with variable elasticity and dimension. This Dresswear, rendered as a digital wire mesh, is not only fitted to the dimensions of the captured skin embodiment but is further re-rendered with zoning of the digital Dresswear with fabrics of variable elasticity (and dimension) to provide selective contouring of the person's own 3D embodiment. For this purpose, contouring zones are incorporated into the custom garment in a seamless fashion. Examples of this rendering and selective zoning of the custom garment may include an incorporated bra zone to provide lift and 3D contouring of the breast. Another example is an incorporated girdle zone that will lift hips and contour-in the waistline. FIG. 5 shows the wire mesh corresponding to the digital 3D capture (left), and the wire mesh with selective zoning in the bra zone and girdle zone (right), under an embodiment. FIG. 6 shows the digital 3D capture of a body without the wire mesh garment (left), and the 3D capture of the body with the wire mesh with selective zoning in the bra zone and girdle zone (right), under an embodiment.

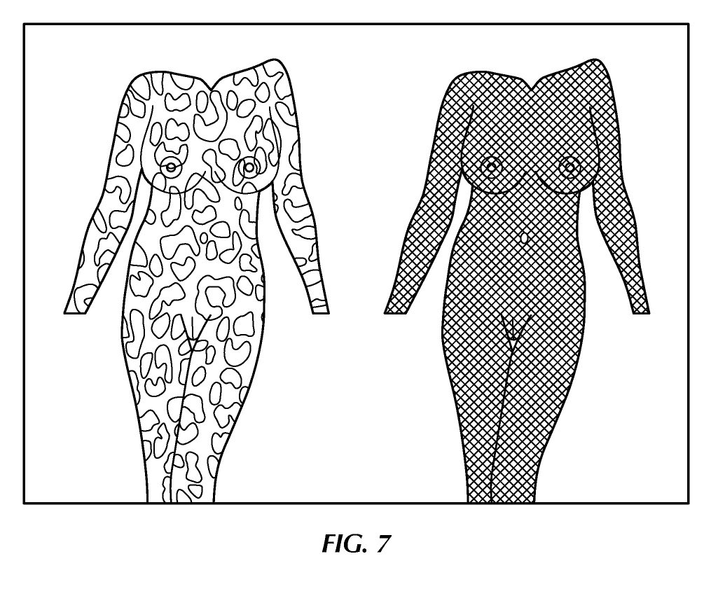

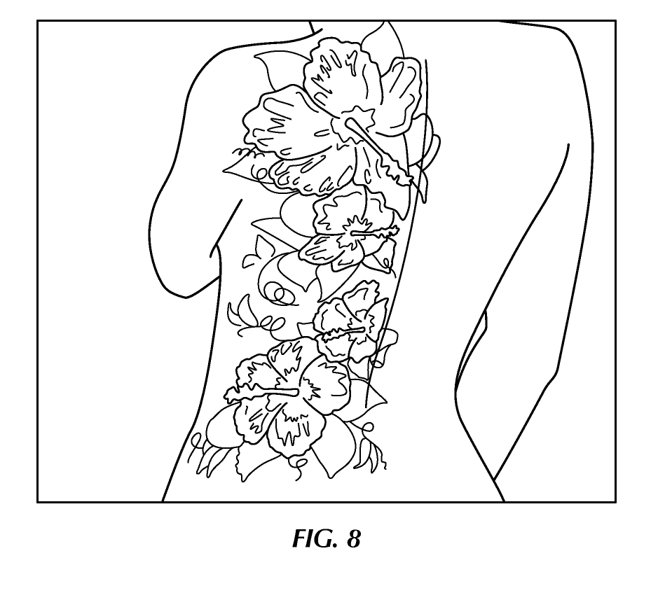

A personal database is generated based on or using digital data of the subject's own skin/soft tissue embodiment. The personal database includes data to provide contour correction. For this purpose, the custom Skinwear is also zoned with mosaic of different fabrics with different elasticities and dimensions. With digital rendering and re-rendering, a custom patterned Skinwear can also be synthetically fabricated to exact dimensions of all body contours. Used primarily as a fashion statement, a custom fitted and patterned Skinwear also provides camouflage to skin and soft tissue deformities of the patient's embodiment. FIG. 7 shows the body with the Skinwear with camouflage (left), and the body with the Skinwear without camouflage (right), under an embodiment.

The Custom Skinwear provides inward contouring and outward expansion into and onto all anatomical regions of the person's skin/soft tissue embodiment. The Skinwear is also fabricated with contouring zones that will seamlessly correct aesthetic contours of the embodiment. This contour correcting Skinwear can also comprise synthetic fabrics of variable translucency including a completely translucent fabric.

A data set for each person for which the digitally captured, wire mesh rendered body contour is then re-rendered as a wire mesh representing a corrected contour. The corrected contour is re-rendered into a zoned digital fabric mosaic to create the corrected contour including the specifications for fabric materials and dimensional specifications in X-Y and Z axis where the X-Y axis is the same throughout for seamless appearance and the z axis (depth) can be variable to provide a selected elasticity for inward and outward contouring. The entire data set is entered into programmable fabricator of synthetic garments that can be programmed for each person's skin/soft tissue embodiment. Further resolution of the dataset is gained with a topographical mapping of the person's own skin elasticity and deformability of the soft tissue component within the person's embodiment i.e., for a more accurate Skinwear fabrication, the data set may include specifications for both the synthetic fabrication and the skin embodiment of the person.

Patterning of the Skinwear is also provided in a programmable fashion to camouflage skin wrinkling caused by aging and/or scar deformities such as burns. A series of renderings and synthetic fabrications may also be used for the most aesthetically enhanced custom fitting of either the Dresswear or the Skinwear. The base color of the Skinwear can also vary depending upon or according to the pigmentation and/or pigmentary pattern of the individual's skin. In an embodiment, the base Skinwear approximates the pigmentary and freckle pattern of the person's skin envelope. The Skinwear appears as a continuation of the patient's own integument. In an alternative embodiment, the Skinwear is more of a fashion statement and is configured to vary from the individual's skin pigmentary pattern but is fashion coordinated to create a complimentary de-novo appearance to the person's skin embodiment.

Yet another embodiment uses a camouflage pattern that is either a geometric or animal print pattern that will distract the observer's eye from an underlying deformity or the pattern becomes a patterned fashion statement to be worn by the person in combination with a more loosely fitting secondary garment that is semitransparent. For example, the animal print acts as camouflage for wrinkled skin and scarring. Additional camouflaging by the Skinwear of an aesthetic contour deformity can also be achieved by selective two-dimensional shading of the Skinwear around and over the deformity i.e., Boundary Camouflaging.

Skin colors embodied in the Skinwear can be variable depending upon the preference of the subjects (e.g., woman's, etc.) choices in fashion and does not need to match her skin color or skin pigmentary pattern. For contouring, the design software topographically depicts on a digital wire mesh, the specific dimensions of zoning of the Skinwear that provides seamless inward and outward contouring. Zone components may comprise different types or compositions of materials, combinations of materials and/or different elasticity of the same fabric due to “Z” axis deposition or layering. The constituent synthetic fabrics can be the same in each zone or uniquely blended or layered depending upon appearance and contouring capabilities. The constituent fabrics (e.g., Rayon, Spandex, Polyester, etc.) are combined to provide less elasticity in areas of inward contouring and more elasticity in areas for expansion needed for outward contouring. A gradation of an embodiment between less elastic fabric and more elastic fibers provides a more gradual shaping of the contour. A component digital wire mesh is created that appears seamless and uniform in its weave pattern and density with other zones but is fully contouring to the person's skin embodiment. This base line digital mesh is re-rendered into a corrected wire mesh that provides additional aesthetic contouring of the person's skin/soft tissue embodiment.

The re-rendered digital wire mesh is mapped onto the patient's skin embodiment for final approval prior to fabrication. The Skinwear comprises the entire body or, alternatively, comprises a region of skin embodiment such as the upper arms, legs, hips, trunk, breasts, etc. For inward contouring into anatomical clefts such as the vertical midline gluteal cleft, the tightness and inflexibility of the fabric is obtained by altering the component (or combination of materials) fabric material or thickening the constituent fabric in the depth (Z) axis. The overall appearance is a seamless conforming garment that fully conforms to all anatomical contours and actively shapes all anatomical regions into more aesthetically enhanced contours.

The different zones of the Skinwear garment of an embodiment are relatively more contouring and/or directionally vectored than other areas on the skin/soft tissue embodiment. Examples of vectoring include an incorporated bra component that may or may not have the same weave pattern as the rest of the garment but is structured to provide not just 3D contouring but provide linear direction (vectoring) for the repositioning of the soft tissue structure into a more aesthetically enhanced position.

At transition margins onto the person's own skin embodiment, the Skinwear of an embodiment includes properties configured to fade into the skin color, pattern and/or texture of the subject. The Skinwear either fades in color/pattern opacity (becoming more transparent) or becomes more closely colored and patterned to the patient's own skin tone and pigmentary pattern, for example. The transition margins are placed at normally-occurring anatomical borders such as the clavicular margin, the wrist and ankle, but are not so limited.

The use of transparent skin adhesives or skin adhesives (with or without similar weave pattern as the synthetic Skinwear) provides additional inward contouring and adherence of the garment at transition areas where the Skinwear and the skin intersect.

FIG. 8 shows custom patterned Skinwear, under an embodiment. The custom patterned Skinwear can be used to camouflage portions of the underlying skin because it is configured to resemble an extension of the pattern of pre-exiting skin tattoos. As such, the Skinwear tattoo pattern appears as an addition to the pre-existing tattoo and negates the need for the subject to need additional skin tattooing for the “fashion effect” of a full body tattoo.

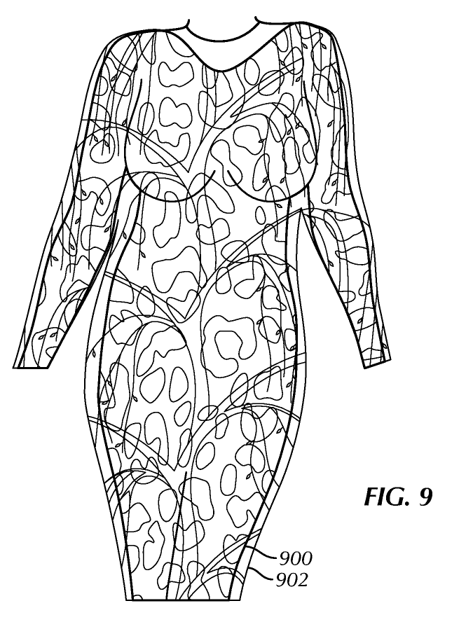

FIG. 9 shows a custom patterned Skinwear ensemble, under an embodiment. The patterned Skinwear ensemble is apparel comprising a loosely fitted variable semitransparent garment 902 configured for wear over patterned Skinwear 900, where the pattern of the Skinwear is visible through the semi-transparent garment.

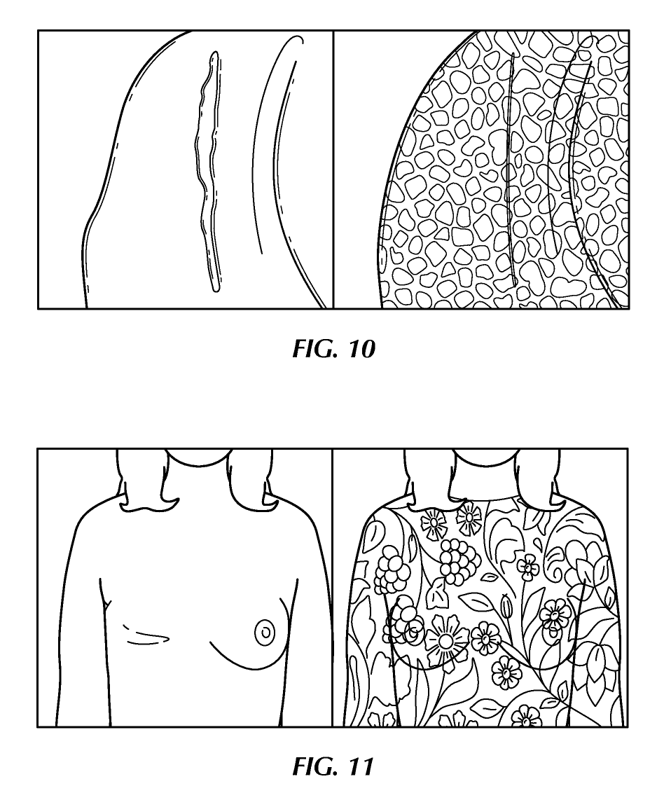

The Skinwear of embodiments is configured for reconstructive applications, for example applications involving traumatic scar and kinetic contour deformities, and post-mastectomy chest deformities as described herein. FIG. 10 shows custom patterned Skinwear (right) configured for applications involving traumatic scar and kinetic contour deformities (left), under an embodiment. In addition to masking unsightly scarring, the Skinwear is configured to contour defects caused by fat necrosis can also occur from blunt kinetic injuries. Many of these deformities are caused by motor vehicular accidents, for example. The use of Custom Camouflage Compression Garments for the treatment of scarring also softens cutaneous scarring. In addition, the custom fabrication of a soft tissue prosthesis (that has a similar viscosity as the adjacent soft tissue) can be incorporated into the compression garment of embodiments.

FIG. 11 shows custom patterned Skinwear (right) configured for applications involving post mastectomy chest deformities (left), under an embodiment. These unsightly and emotionally impactful symmetry deviations of the female embodiment can be mitigated with the fabrication of custom Skinwear that includes an external breast prosthesis. With the local recurrence increasing with lumpectomy and radiation, the need for a subsequent mastectomy has resulted in severe chest deformities that are difficult to reconstruct. The fabrication of a custom external breast prosthesis of an embodiment offers new hope for these patients. The prosthesis can be worn separately or incorporated within a patterned Skinwear that de-delineates the margin of the prosthesis. Similar pattern camouflaging on the surface of the custom prosthesis is also included in an embodiment.

The development of externally worn prosthesis also conforms in contour, color, pigmentary pattern, and texture to the adjacent skin embodiment. The prosthesis of an embodiment is further de-delineated from the surrounding chest skin by digitally capturing the skin pigment color, skin pigmentary pattern and perceived texture of the adjacent skin. The digital capture of this data set is then programed into a 3D printer for application onto the surface of the prosthesis. An embodiment additionally includes the use of an “insitu” material on the surface of the prosthesis that is reprogrammable to match the color and pigmentary pattern changes with time of the adjacent skin of the patient. In addition, the breast contour of the remaining breast can be digitally captured and mirror image re-rendered for symmetric fabrication of the prosthesis. The viscosity/cohesiveness and deformability (as a function of both surface elasticity and viscosity) of the prosthesis can also be custom fabricated for symmetry with the remaining breast.

Additional clinical indications of the Skinwear of an embodiment include applications configured for use with severe burn scarring, vitiligo depigmentation, large congenital nevi, large congenital hemanglomas, and neurofibromatosis, to name a few. Regarding custom Skinwear compressive garment configured for applications involving severe burn scarring, deep second and third degree burns can cause severe scaring that can also limit excursion of limbs across joints such as the axilla. The use of a Skinwear compressive garment of an embodiment camouflages severe burn scarring and also improves range of motion across joints by softening the scaring with compression.

Vitiligo depigmentation is caused by the irregular loss of melanocytes that form skin pigment. The condition results in irregularity in skin pigmentation and is more visible in more deeply pigmented ethnic groups. Custom Skinwear of an embodiment is configured for applications involving vitiligo depigmentation.

Custom Skinwear of an embodiment is configured for applications involving large congenital nevi. Present at birth, many nevi are smaller and more easily amendable for surgical excision. There are two primary indications for surgical excision including removing the visual stigma of the nevus, and reducing the potential for malignant degeneration of the congenital nevus into melanoma which may occur in up to 25 percent of children born with a giant congenital nevus. Due to the significant potential for malignant degeneration, it is recommended that these “giant” lesions be resected. For most patients, this is major multistage process that involves significant scarring associated with the use of skin grafting. Many of these children are severely scarred as a result of these procedures. A custom compression Skinwear product of an embodiment camouflages and softens the extensive scarring associated with the surgical resection and skin grafitng of these large congenital lesions. The use of a Skinwear garment of an embodiment ameliorates the emotional impact of these severe surgically related deformities.

Custom Skinwear of an embodiment is configured for applications involving large congenital Hemangioma. The emotional impact of a large congenital Hemangioma can be as crippling as with a giant congenital Nevus. However, the oncologic risk is not as severe and most of these dermal vascular lesions do not need to be resected. Many of the smaller “strawberry” Hemangiomas will spontaneous involute with time. The larger capillary port wine stain Hemangiomas will not involute with time but will remain a permanent visible deformity. The use of custom patterned Skinwear of an embodiment reduces the visual impact of these large “Port wine Stains”.

The management of large congenital Hemangioma is only a single example of this programmable capability. Skinwear embodiments include and provide direct application of non-toxic synthetic materials directly onto the patient's integument. Following the digital capture and re-rending of dyspigmentary irregularities, a programmable capability allows the topographical spraying or stenciling of a nontoxic mosaic of chromophores in a per patient programmable fashion that enables either pattern camouflaging or de-delineated blending into non-dyspigmentated areas of the skin envelope. Optical mouse mapping and recognition may provide the necessary real time tracking necessary for accurate topographic deposition of pigment materials. The stencil deposition or spraying of the chromophores is performed in pattern that simulates adjacent normal skin pattern and texture.

Custom Skinwear of an embodiment is configured for applications involving neurofibromatosis. This disease of the myelin sheath of nerves is a genetically inherited disease with variable expression from an autosomal dominant trait. Two major classifications have been identified. Type 1 neurofibromatosis may become a visible deformity over a broad surface area of the patient's integument. This phenotypic manifestation is the most common subgroup of the disease. Type 1 Neurofibromatosis is also referred to as Von Recklinghausen disease or peripheral neurofibromatosis. The use of a custom Skinwear garment of an embodiment potentially reduces the visible stigmata associated with this disease. Although not established by a protocol-led study, the continual application of a compression Skinwear garment may also reduce the occurrence and/or mitigate the continual growth of these soft tissue tumors.

Embodiments are configured for applications involving a subject with facial palsy. Embodiments include a micro-elastic fabric of variable elasticity that mimics facial expression at rest where the synthetic Skinwear recreates normal undistorted facial features. The animation of the facial Skinwear is provided by a conductive fiber within the Skinwear that actuates a mechanically contractile fiber within the garment.

Skinwear embodiments include surface contour shading of the Skinwear to reduce the visibility of any contour embodiment asymmetry. Further, Skinwear embodiments include the use of meta-materials (or other programmable cloaking materials) for altering the color, brightness, pigmentary pattern and texture based on the adjacent (or subjacent) surface properties of the skin. The use of Skinwear over years provides a preventive capability that counteracts the prolonged effect of gravity in causing dependent laxity of the skin.

The Skinwear or Dresswear of embodiments includes constituent fabrics having varying properties. An example embodiment of synthetic Dresswear fabric includes Rayon 97% and Spandex 3%, giving the Dresswear the property of a slightly heavier feel. Another example embodiment of synthetic Dresswear fabric includes Polyester 95% and Spandex 5%, giving the Dresswear the property of higher flexibility.

In order to determine the material properties of an appropriate constituent material/polymer for use in a particular Skinwear application, key material properties of polymer should be identified. These properties include but are not limited to composition and structure, melting point, modulus, elasticity and recovery from strain, tensile, density, moisture absorption, dye-ability, and comfort. Regarding composition and structure, the type of polymer, the amount/length of side chains, the shape of the polymer, and the shape and amount/length of side chains play a role in the strength of the polymer.

The melting point is the temperature at which the polymer begins to melt. Melt strength is a property that indicates the polymer's ability to withstand drawing without breaking. This is improved with the presence of high molecular weight tail or long chain branches. Modulus is the ability of a material to resist deformation, and this property is generally expressed as the ratio of stress exerted on the sample to the amount of deformation.

Elasticity and recovery from strain are considered. The elastic limit is the stress at which the uniaxial stress-strain curve becomes nonlinear due to shear yielding or crazing. Shear yielding is the irreversible slipping of molecule chairs, and crazing is the formation of low density, crack-like volumes which scatter light (makes the polymer appear white).

Tensile properties are the most important indication of strength and stiffness of the material. They determine the force necessary to pull the specimen apart, and the deformation before breaking. The tensile modulus is a measure of stiffness calculated based on measured force.

Density, or the weight per unit volume, affects physical properties like stiffness, impact strength, and optical properties. Moisture absorption is the amount of moisture a fiber will absorb from the air at a standard condition of 70° F. and a relative humidity of 65%. Dye-ability is the ability of the polymer to absorb and hold on to a dye.

Comfort is a measure of how the material feels to the user. If the fiber or fabric is going to be used in apparel then the comfort stretch (freedom of movement when wearing the fabric), phase change ability, and hydrophobic nature are considered.

Constituent materials of the Skinwear or Dresswear include any of a variety of material types or compositions, and these various materials are referred to using terms described herein. For example the terms “fabric” and “cloth” are used in textile assembly trades (e.g., tailoring and dressmaking) as synonyms for “textile”. However, there are subtle differences in these terms in specialized usage. “Textile” refers to any material made of interlacing fibers. “Fabric” refers to any material made through weaving, knitting, spreading, crocheting, or bonding that may be used in production of further goods (garments, etc.). “Cloth” may be used synonymously with “fabric” but often refers to a finished piece of fabric used for a specific purpose.

Brand names and composition of synthetic textiles capable of use in the production of Skinwear include polyester or polyester fiber, aramid fiber, acrylic, nylon, spandex, olefin, ingeo, lurex, and carbon fiber to name a few. Polyester refers to a category of polymers used in all types of clothing, either alone or blended with natural fibers such as cotton. Aramid fiber (e.g. Twaron) is used for flame-retardant clothing, cut-protection, and armor. Acrylic is a fiber used to imitate wools, including cashmere, and is often used in replacement of them. Nylon is a fiber used to imitate silk; it is used in the production of pantyhose. Thicker nylon fiber are used in rope and outdoor clothing.

Spandex (trade name Lycra) is a polyester-polyurethane copolymer product that can be made tight-fitting without impeding movement, and is used to make activewear, bras, and swimsuits. Olefin fiber is a fiber used in activewear, linings, and warm clothing. Olefins are hydrophobic, allowing them to dry quickly. A sintered felt of olefin fibers is sold under the trade name Tyvek. Ingeo is a polylactide fiber blended with other fibers such as cotton and used in clothing, and is more hydrophilic than most other synthetics, allowing it to wick away perspiration.

Rayon is a semi-synthetic fiber manufactured from naturally occurring polymers. Lurex is a metallic fiber used in clothing embellishment. Carbon Fiber is mostly used in composite materials, together with resin, such as carbon fiber reinforced plastic. The fiber is made from polymer fibers through carbonization.

The constituent materials of embodiments include weave patterns. Synthetic fibers and synthetic fabrics comprise bulk fibers, yams, woven cloth or other textile products manufactured from polymer-based materials such as polyamide (nylon), polyester, aramid, or other spun thermoplastics. The resulting product is the form of the fiber/fabric when manufacturing is complete. Basic product types include, but are not limited to, fibers and monofilaments, roving, yarn, carded and needle punched (non-woven), braided, rope and cordage, webbing, blankets or batts, sleeves or wraps, and thread to name a few.

Single fibers are called filaments and a monofilament is when a single continuous filament is rolled on a spool. A filament bunch is called a strand or end. Bulk chopped fibers or thin, continuous fiber filaments are used typically in composite reinforcement applications, flow-able insulation, or as the key component in woven fabrics, braids, knits, rope roving, or other specialty fabrics.

Roving is made of parallel filaments. Graphite rovings are referred to as tows. Rovings are marked according to the number of included filaments. Tows are marked according to the number of filaments, with common graphite tows being 3K, 6K and 12K.

Yarn comprises continuous, often plied strands of natural or man-made fibers or filaments. The filament is then twisted to hold fibers together.

Regarding carded and needle punched non-woven material, carded yarn has been through the card machine but has not been combed. Because they include a range of fiber lengths they are low strength, low density, and low cost.

Braided fabrics are made by crossing a number of strands diagonally so each strand passed alternating over or under one or more of the other strands. These products are used for tubular composite structures, thermal insulation, and in other applications.

Cord is formed by twisting multiple plied yarns, and can also be defined as a rib on the surface of a fabric. Rope is a heavier and stronger cord made from either natural or synthetic fibers and is available in a wide range of diameters. Rope is made in a process in which the yarns are twisted together to form strands, and then the strands are twisted together in the opposite direction to form the rope. The alternating direction of the twist at different stages of the rope assembly makes the rope twist stable and resistance to kinks.

Webbing is strong, narrow fabric that has been closely woven. Webbing is available in a variety of weaves and often found in straps that have to withstand strain (belt, seat belts, suspenders, etc.). Webbing includes ribbons, strapping, and tape.

Blankets or batts (batting) are made of thick layers of woven and/or non-woven fabric sheets. Battings includes webs of loose fibers that have usually been carded. Battings are sold in sheets or rolls and used for warm linings and comforter filling.

Sleeves or wraps (sleeving) are flexible, fibrous refractory products for insulating pipes, tubes, ducts, and other process components.

Synthetic thread includes both monofilaments and multi-fiber filament; a slender, strong strand or cord. Most threads are made by plying and twisting yarns but are not so limited. There are a large variety of yarns available for many different industrial applications.

Most synthetic fibers go through a similar production process, the steps of which are generally described herein. A chemical process, usually polymerization, prepares and combines the components for the fiber. Polymerization is the formation of macromolecules through repetition of basic units. Initially, the various components are solids and first must be converted to a liquid state to be extruded into fibers. The materials are chemically converted, dissolved, or melted, turning into a thick liquid.

A spinning process produces the fiber by passing the thick liquid through a spinneret. A spinneret is a device with hundreds of holes of a specified diameter. The liquid is forced through the spinneret holes and comes out a string liquid filament. The hole in the spinneret determines the diameter of the filament, which is set by the application. The extrusion is dried to a continuous filament fiber.

A twisting process twists the filament fiber into a yarn. The filament falls vertically from the spinneret and is caught in a large vacuum nozzle. The vacuum force keeps tension on the line as it is wound around a bobbin.

The twisted yarn is packaged and sent to a textile mill.

Embodiments include a method comprising capturing digital data representing skin of a body. The method generates from the digital data an avatar representing the skin of the body. The method generates an enhanced avatar by digitally revising a contour of the skin of the body. The method renders from the enhanced avatar a digital garment configured to enhance contours of the skin of the body. The digital garment comprises material having a plurality of zones, and each zone is configured with material comprising at least one of a contouring force and a pigment pattern corresponding to a region of the skin corresponding to the zone. The method generates from the digital garment a corrective garment configured to be worn to cover at least one region of the skin of the body. The corrective garment is configured to aesthetically enhance the body.

Embodiments include a method comprising: capturing digital data representing skin of a body; generating from the digital data an avatar representing the skin of the body; generating an enhanced avatar by digitally revising a contour of the skin of the body; rendering from the enhanced avatar a digital garment configured to enhance contours of the skin of the body, wherein the digital garment comprises material having a plurality of zones, wherein each zone is configured with material comprising at least one of a contouring force and a pigment pattern corresponding to a region of the skin corresponding to the zone; generating from the digital garment a corrective garment configured to be worn to cover at least one region of the skin of the body, wherein the corrective garment is configured to aesthetically enhance the body.

The method comprises generating a database comprising the digital data, avatar data, enhanced avatar data, the digital garment, and corrective garment data.

The corrective garment is configured to deliver the contouring force with the material instead of seaming.

The contouring force vectors and conforms the skin to generate the aesthetically enhanced body.

Transition margins between the plurality of zones are located to correspond to anatomical borders of the body.

At least one of the avatar and the enhanced avatar comprise a wire mesh object digitally rendered from the digital data representing the skin of the body.

At least one of the avatar, the enhanced avatar, the digital garment, and the corrective garment comprise three-dimensional representations.

At least one of the avatar, the enhanced avatar, the digital garment, and the corrective garment comprise four-dimensional representations.

The capturing of the digital data comprises capturing a digital image of the body.

The digital data comprises data of physical properties of at least one of the skin and soft tissue underlying the skin.

The digital data comprises at least one of pigment data, pigment pattern data, tone data, color data, texture data, elasticity data, laxity data, deformity data, wrinkling data, scar data, and burn data of at least one of the skin and soft tissue underlying the skin.

The method comprises collecting the data of physical properties using at least one medical device.

Each zone is configured with material comprising at least one characteristic corresponding to a region of the skin corresponding to the zone.

The corrective garment comprises an appearance of a continuation of the skin of the body.

The corrective garment comprises a camouflage pattern configured to mask an underlying deformity of the skin.

The underlying deformity includes at least one of a scar, tattoo, wrinkle, skin laxity, and contour deformity.

The underlying deformity includes at least one of a breast deformity and a mastectomy deformity.

The underlying deformity includes a contour scar deformity caused by at least one of a high kinetic injury and a burn.

The plurality of zones include a bra zone configured to provide at least one of lift and contouring of a breast of the body.

The plurality of zones include a girdle zone configured to provide at least one of lift and contouring of at least one of a hip and waist of the body.

The aesthetic enhancement includes seamless correction of at least one of aesthetic contours, soft tissue deformities, and skin deformities of the body.

The method comprises generating the digital garment to include synthetic material.

The method comprises generating at least one zone to include at least of a size and a synthetic material that is different from at least one other size and synthetic material of at least one other zone.

The method comprises generating the plurality of zones of the material to include variable flexibility.

The method comprises generating at least one zone to include at least one synthetic material having a flexibility different from another flexibility of at least one other synthetic material of at least one other zone.

The method comprises providing the variable flexibility by using different synthetic materials among different zones of the plurality of zones.

The method comprises providing the variable flexibility by using, among different zones of the plurality of zones, materials having different thicknesses.

The contouring force comprises outward contouring, and the material of a zone corresponding to the outward contouring comprises parameters providing increased flexibility and outward expansion.

The contouring force comprises inward contouring, and the material of a zone corresponding to the inward contouring comprises parameters providing decreased flexibility.

The method comprises orienting the zone corresponding to the inward contouring over at least one anatomical cleft.

The at least one anatomical cleft includes at least one of a vertical midline gluteal cleft and a horizontal inframammary fold.

The corrective garment comprises an outer garment configured for wear over the corrective garment, wherein the outer garment is configured as semi-transparent.

The corrective garment comprises a prosthesis.

Te material of the corrective garment includes at least one of thread, fiber, filament, woven material, yarn, braided material, cords, batts, sleeves, Rayon, Spandex, Polyester, Acrylic, Aramid, Nylon, Olefin, Ingeo, and Lurex.

Embodiments include a system comprising a processor coupled to a memory. A detector is coupled to the processor and configured to capture digital data representing skin of a body. A rendering application executes on the processor. The rendering application is configured to generate from the digital data an avatar representing the skin of the body, generate an enhanced avatar by digitally revising a contour of the skin of the body, and render from the enhanced avatar a digital garment configured to enhance contours of the skin of the body. The digital garment comprises material having a plurality of zones. Each zone is configured with material comprising at least one of a contouring force and a pigment pattern corresponding to a region of the skin corresponding to the zone. A garment generator is coupled to the processor and configured to receive the digital garment and generate from the digital garment a corrective garment configured to be worn to cover at least one region of the skin of the body. The corrective garment is configured to aesthetically enhance the body.

Embodiments include a system comprising: a processor coupled to a memory; a detector coupled to the processor and configured to capture digital data representing skin of a body; a rendering application executing on the processor, wherein the rendering application is configured to generate from the digital data an avatar representing the skin of the body, generate an enhanced avatar by digitally revising a contour of the skin of the body, and render from the enhanced avatar a digital garment configured to enhance contours of the skin of the body, wherein the digital garment comprises material having a plurality of zones, wherein each zone is configured with material comprising at least one of a contouring force and a pigment pattern corresponding to a region of the skin corresponding to the zone; and a garment generator coupled to the processor and configured to receive the digital garment and generate from the digital garment a corrective garment configured to be worn to cover at least one region of the skin of the body, wherein the corrective garment is configured to aesthetically enhance the body.

Embodiments include a system comprising a processor coupled to a memory. A detector is coupled to the processor and configured to capture digital data representing skin of a body. A rendering application executes on the processor. The rendering application is configured to generate from the digital data an avatar representing the skin of the body, generate an enhanced avatar by digitally revising a contour of the skin of the body, and render from the enhanced avatar a digital garment configured to enhance contours of the skin of the body. The digital garment comprises material having a plurality of zones. Each zone is configured with material comprising at least one of a contouring force and a pigment pattern corresponding to a region of the skin corresponding to the zone. A corrective garment is generated from the digital garment. The corrective garment is configured to be worn to cover at least one region of the skin of the body. The corrective garment is configured to aesthetically enhance the body.

Embodiments include a system comprising: a processor coupled to a memory; a detector coupled to the processor and configured to capture digital data representing skin of a body; and a rendering application executing on the processor, wherein the rendering application is configured to generate from the digital data an avatar representing the skin of the body, generate an enhanced avatar by digitally revising a contour of the skin of the body, and render from the enhanced avatar a digital garment configured to enhance contours of the skin of the body, wherein the digital garment comprises material having a plurality of zones, wherein each zone is configured with material comprising at least one of a contouring force and a pigment pattern corresponding to a region of the skin corresponding to the zone, wherein a corrective garment is generated from the digital garment, wherein the corrective garment is configured to be worn to cover at least one region of the skin of the body, wherein the corrective garment is configured to aesthetically enhance the body.

Embodiments include a method comprising capturing digital data representing skin of a body. The method comprises generating from the digital data an avatar representing the skin of the body. The method comprises rendering from the avatar a digital garment configured to match contours of the skin of the body. The method comprises generating from the digital garment a custom-fitted garment configured to be worn by the body.

Embodiments include a method comprising: capturing digital data representing skin of a body; generating from the digital data an avatar representing the skin of the body; rendering from the avatar a digital garment configured to match contours of the skin of the body; and generating from the digital garment a custom-fitted garment configured to be worn by the body.

Embodiments include a system comprising a processor coupled to a memory. A detector is coupled to the processor and configured to capture digital data representing skin of a body. A rendering application executes on the processor. The rendering application is configured to generate from the digital data an avatar representing the skin of the body, and render from the avatar a digital garment configured to match contours of the skin of the body. A garment generator is coupled to the processor and configured to receive the digital garment and generate from the digital garment a custom-fitted garment configured to be worn by the body.

Embodiments include a system comprising: a processor coupled to a memory; a detector coupled to the processor and configured to capture digital data representing skin of a body; a rendering application executing on the processor, wherein the rendering application is configured to generate from the digital data an avatar representing the skin of the body, and render from the avatar a digital garment configured to match contours of the skin of the body; and a garment generator coupled to the processor and configured to receive the digital garment and generate from the digital garment a custom-fitted garment configured to be worn by the body.

Embodiments include a system comprising a processor coupled to a memory. A detector is coupled to the processor and configured to capture digital data representing skin of a body. A rendering application executes on the processor. The rendering application is configured to generate from the digital data an avatar representing the skin of the body, and render from the avatar a digital garment configured to match contours of the skin of the body. A custom-fitted garment is generated from the digital garment. The custom-fitted garment is configured to be worn by the body.

Embodiments include a system comprising: a processor coupled to a memory; a detector coupled to the processor and configured to capture digital data representing skin of a body; and a rendering application executing on the processor, wherein the rendering application is configured to generate from the digital data an avatar representing the skin of the body, render from the avatar a digital garment configured to match contours of the skin of the body, wherein a custom-fitted garment is generated from the digital garment, wherein the custom-fitted garment is configured to be worn by the body.

Computers, processors, and networks suitable for use with the embodiments described herein include local area networks (LAN), wide area networks (WAN), Internet, or other connection services and network variations such as the world wide web, the public internet, a private internet, a private computer network, a public network, a mobile network, a cellular network, a value-added network, and the like. Computing devices coupled or connected to the network may be any microprocessor controlled device that permits access to the network, including terminal devices, such as personal computers, workstations, servers, mini computers, main-frame computers, laptop computers, mobile computers, palm top computers, hand held computers, mobile phones, TV set-top boxes, or combinations thereof. The computer network may include one of more LANs, WANs, Internets, and computers. The computers may serve as servers, clients, or a combination thereof.

The system can be a component of a single system, multiple systems, and/or geographically separate systems. The system can also be a subcomponent or subsystem of a single system, multiple systems, and/or geographically separate systems. The system can be coupled to one or more other components (not shown) of a host system or a system coupled to the host system.

One or more components of the system and/or a corresponding system or application to which the system is coupled or connected include and/or run under and/or in association with a processing system. The processing system includes any collection of processor-based devices or computing devices operating together, or components of processing systems or devices, as is known in the art. For example, the processing system can include one or more of a portable computer, portable communication device operating in a communication network, and/or a network server. The portable computer can be any of a number and/or combination of devices selected from among personal computers, personal digital assistants, portable computing devices, and portable communication devices, but is not so limited. The processing system can include components within a larger computer system.

The processing system of an embodiment includes at least one processor and at least one memory device or subsystem. The processing system can also include or be coupled to at least one database. The term “processor” as generally used herein refers to any logic processing unit, such as one or more central processing units (CPUs), digital signal processors (DSPs), application-specific integrated circuits (ASIC), etc. The processor and memory can be monolithically integrated onto a single chip, distributed among a number of chips or components, and/or provided by some combination of algorithms. The methods described herein can be implemented in one or more of software algorithm(s), programs, firmware, hardware, components, circuitry, in any combination.

The components of any system that includes the system can be located together or in separate locations. Communication paths couple the components and include any medium for communicating or transferring files among the components. The communication paths include wireless connections, wired connections, and hybrid wireless/wired connections. The communication paths also include couplings or connections to networks including local area networks (LANs), metropolitan area networks (MANs), wide area networks (WANs), proprietary networks, interoffice or backend networks, and the Internet. Furthermore, the communication paths include removable fixed mediums like floppy disks, hard disk drives, and CD-ROM disks, as well as flash RAM, Universal Serial Bus (USB) connections, RS-232 connections, telephone lines, buses, and electronic mail messages.

Aspects of the system and corresponding systems and methods described herein may be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the system and corresponding systems and methods include: microcontrollers with memory (such as electronically erasable programmable read only memory (EEPROM)), embedded microprocessors, firmware, software, etc. Furthermore, aspects of the system and corresponding systems and methods may be embodied in microprocessors having software-based circuit emulation, discrete logic (sequential and combinatorial), custom devices, fuzzy (neural) logic, quantum devices, and hybrids of any of the above device types. Of course the underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

It should be noted that any system, method, and/or other components disclosed herein may be described using computer aided design tools and expressed (or represented), as data and/or instructions embodied in various computer-readable media, in terms of their behavioral, register transfer, logic component, transistor, layout geometries, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, etc.). When received within a computer system via one or more computer-readable media, such data and/or instruction-based expressions of the above described components may be processed by a processing entity (e.g., one or more processors) within the computer system in conjunction with execution of one or more other computer programs.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import, when used in this application, refer to this application as a whole and not to any particular portions of this application. When the word “or” is used in reference to a list of two or more items, that word covers all of the following interpretations of the word: any of the items in the list, all of the items in the list and any combination of the items in the list.

The above description of embodiments of the system and corresponding systems and methods is not intended to be exhaustive or to limit the systems and methods to the precise forms disclosed. While specific embodiments of, and examples for, the system and corresponding systems and methods are described herein for illustrative purposes, various equivalent modifications are possible within the scope of the systems and methods, as those skilled in the relevant art will recognize. The teachings of the system and corresponding systems and methods provided herein can be applied to other systems and methods, not only for the systems and methods described above.

The elements and acts of the various embodiments described above can be combined to provide further embodiments. These and other changes can be made to the system and corresponding systems and methods in light of the above detailed description.

In general, in the following claims, the terms used should not be construed to limit the system and corresponding systems and methods to the specific embodiments disclosed in the specification and the claims, but should be construed to include all systems that operate under the claims. Accordingly, the system and corresponding systems and methods is not limited by the disclosure, but instead the scope is to be determined entirely by the claims.

While certain aspects of the system and corresponding systems and methods are presented below in certain claim forms, the inventors contemplate the various aspects of the system and corresponding systems and methods in any number of claim forms. Accordingly, the inventors reserve the right to add additional claims after filing the application to pursue such additional claim forms for other aspects of the system and corresponding systems and methods.

What is claimed is:

1. A method comprising:
capturing digital data representing skin of a body;
generating from the digital data an avatar representing the skin of the body;
generating an enhanced avatar by digitally revising a contour of the skin of the body;
rendering from the enhanced avatar a digital garment configured to enhance contours of the skin of the body, wherein the digital garment comprises material having a plurality of zones, wherein each zone is configured with material comprising at least one of a contouring force and a pigment pattern corresponding to a region of the skin corresponding to the zone;
generating from the digital garment a corrective garment configured to be worn to cover at least one region of the skin of the body, wherein the corrective garment is configured to aesthetically enhance the body.

2. The method of claim 1, comprising generating a database comprising the digital data, avatar data, enhanced avatar data, the digital garment, and corrective garment data.

3. The method of claim 1, wherein the corrective garment is configured to deliver the contouring force with the material instead of seaming.

4. The method of claim 3, wherein the contouring force vectors and conforms the skin to generate the aesthetically enhanced body.

5. The method of claim 1, wherein transition margins between the plurality of zones are located to correspond to anatomical borders of the body.

6. The method of claim 1, wherein at least one of the avatar and the enhanced avatar comprise a wire mesh object digitally rendered from the digital data representing the skin of the body.

7. The method of claim 1, wherein at least one of the avatar, the enhanced avatar, the digital garment, and the corrective garment comprise three-dimensional representations.

8. The method of claim 1, wherein at least one of the avatar, the enhanced avatar, the digital garment, and the corrective garment comprise four-dimensional representations.

9. The method of claim 1, wherein the capturing of the digital data comprises capturing a digital image of the body.

10. The method of claim 1, wherein the digital data comprises data of physical properties of at least one of the skin and soft tissue underlying the skin.

11. The method of claim 10, wherein the digital data comprises at least one of pigment data, pigment pattern data, tone data, color data, texture data, elasticity data, laxity data, deformity data, wrinkling data, scar data, and burn data of at least one of the skin and soft tissue underlying the skin.

12. The method of claim 10, comprising collecting the data of physical properties using at least one medical device.

13. The method of claim 1, wherein each zone is configured with material comprising at least one characteristic corresponding to a region of the skin corresponding to the zone.

14. The method of claim 1, wherein the corrective garment comprises an appearance of a continuation of the skin of the body.

15. The method of claim 1, wherein the corrective garment comprises a camouflage pattern configured to mask an underlying deformity of the skin.

16. The method of claim 15, wherein the underlying deformity includes at least one of a scar, tattoo, wrinkle, skin laxity, and contour deformity.

17. The method of claim 15, wherein the underlying deformity includes at least one of a breast deformity and a mastectomy deformity.

18. The method of claim 15, wherein the underlying deformity includes a contour scar deformity caused by at least one of a high kinetic injury and a burn.

19. The method of claim 1, wherein the plurality of zones include a bra zone configured to provide at least one of lift and contouring of a breast of the body.

20. The method of claim 1, wherein the plurality of zones include a girdle zone configured to provide at least one of lift and contouring of at least one of a hip and waist of the body.

21. The method of claim 1, wherein the aesthetic enhancement includes seamless correction of at least one of aesthetic contours, soft tissue deformities, and skin deformities of the body.

22. The method of claim 1, comprising generating the digital garment to include synthetic material.

23. The method of claim 1, comprising generating at least one zone to include at least of a size and a synthetic material that is different from at least one other size and synthetic material of at least one other zone.

24. The method of claim 1, comprising generating the plurality of zones of the material to include variable flexibility.

25. The method of claim 24, comprising generating at least one zone to include at least one synthetic material having a flexibility different from another flexibility of at least one other synthetic material of at least one other zone.

26. The method of claim 24, comprising providing the variable flexibility by using different synthetic materials among different zones of the plurality of zones.

27. The method of claim 24, comprising providing the variable flexibility by using, among different zones of the plurality of zones, materials having different thicknesses.

28. The method of claim 1, wherein the contouring force comprises outward contouring, and the material of a zone corresponding to the outward contouring comprises parameters providing increased flexibility and outward expansion.

29. The method of claim 1, wherein the contouring force comprises inward contouring, and the material of a zone corresponding to the inward contouring comprises parameters providing decreased flexibility.

30. The method of claim 29, comprising orienting the zone corresponding to the inward contouring over at least one anatomical cleft.

31. The method of claim 30, wherein the at least one anatomical cleft includes at least one of a vertical midline gluteal cleft and a horizontal inframammary fold.

32. The method of claim 1, wherein the corrective garment comprises an outer garment configured for wear over the corrective garment, wherein the outer garment is configured as semi-transparent.

33. The method of claim 1, wherein the corrective garment comprises a prosthesis.

34. The method of claim 1, wherein the material of the corrective garment includes at least one of thread, fiber, filament, woven material, yarn, braided material, cords, batts, sleeves, Rayon, Spandex, Polyester, Acrylic, Aramid, Nylon, Olefin, Ingeo, and Lurex.

35. A system comprising:
a processor coupled to a memory;
a detector coupled to the processor and configured to capture digital data representing skin of a body;
a rendering application executing on the processor, wherein the rendering application is configured to generate from the digital data an avatar representing the skin of the body, generate an enhanced avatar by digitally revising a contour of the skin of the body, and render from the enhanced avatar a digital garment configured to enhance contours of the skin of the body, wherein the digital garment comprises material having a plurality of zones, wherein each zone is configured with material comprising at least one of a contouring force and a pigment pattern corresponding to a region of the skin corresponding to the zone; and
a garment generator coupled to the processor and configured to receive the digital garment and generate from the digital garment a corrective garment configured to be worn to cover at least one region of the skin of the body, wherein the corrective garment is configured to aesthetically enhance the body.

36. A system comprising:
a processor coupled to a memory;
a detector coupled to the processor and configured to capture digital data representing skin of a body; and
a rendering application executing on the processor, wherein the rendering application is configured to generate from the digital data an avatar representing the skin of the body, generate an enhanced avatar by digitally revising a contour of the skin of the body, and render from the enhanced avatar a digital garment configured to enhance contours of the skin of the body, wherein the digital garment comprises material having a plurality of zones, wherein each zone is configured with material comprising at least one of a contouring force and a pigment pattern corresponding to a region of the skin corresponding to the zone, wherein a corrective garment is generated from the digital garment, wherein the corrective garment is configured to be worn to cover at least one region of the skin of the body, wherein the corrective garment is configured to aesthetically enhance the body.

37. A method comprising:
capturing digital data representing skin of a body;
generating from the digital data an avatar representing the skin of the body;
rendering from the avatar a digital garment configured to match contours of the skin of the body; and
generating from the digital garment a custom-fitted garment configured to be worn by the body.

38. A system comprising:
a processor coupled to a memory;
a detector coupled to the processor and configured to capture digital data representing skin of a body;
a rendering application executing on the processor, wherein the rendering application is configured to generate from the digital data an avatar representing the skin of the body, and render from the avatar a digital garment configured to match contours of the skin of the body; and
a garment generator coupled to the processor and configured to receive the digital garment and generate from the digital garment a custom-fitted garment configured to be worn by the body.

39. A system comprising:
a processor coupled to a memory;
a detector coupled to the processor and configured to capture digital data representing skin of a body; and
a rendering application executing on the processor, wherein the rendering application is configured to generate from the digital data an avatar representing the skin of the body, render from the avatar a digital garment configured to match contours of the skin of the body, wherein a custom-fitted garment is generated from the digital garment, wherein the custom-fitted garment is configured to be worn by the body.
